(19) United States
(12) Patent Application Publication
(10) Pub. No.: US 2024/0183000 A1
(43) Pub. Date: Jun. 6, 2024

(54) A Biochemical Composition For Increasing Efficiency In Ore Beneficiation and Method of Use Thereof

(71) Applicant: GABIO BIYOLOJIK URUNLER ANONiM SiRKETi, Sisli/ Ankara (TR)

(72) Inventor: Mehmet Sinan OKAY, Besiktas (TR)

(21) Appl. No.: 18/556,302

(22) PCT Filed: Apr. 18, 2022
(86) PCT No.: PCT/TR2022/050344

§ 371 Date: Oct. 19, 2023

(30) Foreign Application Priority Data
Apr. 19, 2021 (TR) 2021/006819

(51) Int. Cl. C22B 1/00; B03B 1/04; B03B 5/00; C12N 1/18

(52) U.S. Cl. CPC C22B 1/00; B03B 1/04; B03B 5/00; C12N 1/18

(57) ABSTRACT

The present invention relates to an environmentally-friendly biochemical composition for use as an additive into an aqueous medium in order to increase the process efficiency and resulting grade in all gravimetric and magnetic wet ore concentration and classification methods that essentially require the use of water, in the mining industry. The invention particularly relates to a biochemical liquid concentrate composition for use as an additive into the existing process or feed water, and a method of use thereof, comprising a fermentation supernatant obtained from the culture of Saccharomyces cerevisiae, one or more surfactants selected from the group consisting of non-ionic surfactants and anionic surfactants, preferably hydrogen peroxide or chlorine dioxide, and urea-based or other suitable preservatives, which increases the process efficiency and grade recovery, without requiring an additional facility investment, in said ore beneficiation processes.

### TECHNICAL FIELD

The present invention relates to a composition for increasing efficiency in ore processing and beneficiation in the mining industry, and a method of use thereof. The invention particularly relates to an environmentally-friendly biochemical composition for use as an additive into the process/feed water in the wet beneficiation processes and a method of use thereof, which provides an increase in grade and yield with ease of operation.

### PRIOR ART

Ore beneficiation process is required and applied for obtaining various minerals from ore in such a way that the value bearing (precious) minerals are separated from those of insignificant economic value without harming chemical structures thereof and thus getting the same as the most suitable raw material needed by the industry. The enrichment (concentration) process involves the separation of valuable minerals/content from the other raw materials, of run-of-the mine ore, wherein the resulting product containing mostly precious minerals is concentrate, and the remaining product of no value is gangue.

The liberation of the precious ore minerals with desired particle size is achieved by using conventional techniques of size reduction (crushing, grinding) and sorting according to size (sieving, classification). After liberation by size reduction and control procedure, a target mineral or material is recovered in the concentrate depending on the properties thereof by various methods of separation. In the classification technique, for instance, particles having different densities with various shapes and sizes are precipitated in a still or flowing fluid medium. Precious minerals can be collected in a single concentrate or obtained in separate concentrates.

For the ore beneficiation processes, methods to be employed are determined according to the physical properties (hardness, brittleness, color or brightness, specific gravity, magnetic susceptibility, fluorescence or phosphorescence properties, radioactivity), physico-chemical properties (surface properties, interface properties) or chemical properties (thermal properties, different solubility) of minerals.

While the large-size minerals are separated, for instance, by sorting by hand based on appearance and color differences or by gravity method based on specific gravity differences among them, ore concentration can be alternatively accomplished through a variety of other methods for fine-grained minerals, including gravity, flotation and selective flocculation. Likewise, magnetic separation based upon natural or induced differences in magnetic susceptibility of the minerals within the ore and chemical separation according to different chemical and dissolution properties thereof are also the methods employed for concentrating ores.

For instance, the principle used in the gravimetric concentration methods is that ore grains are separated in a thin film of fluid (water) flowing over a curved or flat surface. Besides the specific density and speed of the mineral grains in the fluid medium, other specifications of grains such as particle size, particle shape, the density of fluid and viscosity values are important, as well. In a water film flowing on such a surface, the water velocity is not the same everywhere, it approaches zero at the bottom and reaches its highest value on the film. Under these conditions, among the crushed ore grains fed on the surface, the light particles are carried downward, while the heavier ones are separated by being deposited on the surface. Depending on the grain size, heavy media cones and drums, heavy media cyclones, jigs, shaking tables, spirals and pinched sluices can be used in wet processes.

The shaking table is one of the commonly used equipment in beneficiation plants for concentrating minerals based on gravity difference by employing a fluid medium, consisting of a sloping deck with a riffled/ribbed surface across which a film of water flows and is generally rectangular in shape. A longitudinal shaking motion driven by a suitable mechanism consists of a slow forward stroke followed by a quick return strike (back and forth motion) and thus a layered flow is provided on the ribbed surface including parallel bed strips or grooves along the longitudinal direction. With the fluid flow in layers (thin film, shear flow), the particle grains of feed material are separated according to the differences in their density, size and shape, by moving (diagonally) across the deck surface towards the end of the table under the resultant effect of and the asymmetrical movement perpendicular to the flow direction. Due to the fact that the rolling motion is most effective on coarse particles, the lightest-coarsest particle with the fastest movement in the fluid layer takes the shortest path in the direction of the table motion, while the heaviest-finest particle with the slowest movement takes the longest path.

Shaking tables find extensive use in concentrating coal, chromite, cassiterite, tungsten, iron ores other than magnetite, beach sand, tin, lead oxide, zinc, copper, mica, titanium, tantalum, zircon, barite, phosphate, potassium and manganese ores, but are also used in the recovery of gold, silver, thorium and uranium to a lesser extent.

In ore beneficiation processes that require again the use of water but this time rely on magnetic forces, wet magnetic separators are employed, which are broadly categorized into two groups according to the intensity of the magnetic field, namely low-intensity, and high-intensity. Wet high-intensity magnetic separators are mostly used to separate weakly magnetic iron ores such as hematite, limonite, goethite and siderite from non-magnetic or less magnetic gangue materials. Likewise, they are also employed for the separation of ceramic raw materials, glass sand from ferrous minerals and for the enrichment of chromite and manganese ores. On the other hand, wet low-intensity magnetic separators are normally used for the beneficiation of magnetite and for the recovery of ferromagnetic solids (magnetite, ferro-silicon) in heavy medium circuits.

The ore beneficiation method should also be economically viable in addition to all features and requirements mentioned above. It was also a primary concern to make use of more effective and environmentally friendly new technologies for producing higher grade mineral concentrates from run-of-mine materials as non-renewable natural resources are rapidly depleted day by day.

Besides the cost and efficiency considerations in terms of amount, quality and lime content of the water to be used in wet processes, as well as life and maintenance needs of the material onto which it is employed, it is of also great importance to consider the resulting waste and negative effects of the same on living things, the environment and nature.

No prior art teaching has been found about a biochemical composition for use as an additive in to the water in order to increase the efficiency in gravimetric or magnetic concentration methods. It is known, however, that the bioremediation of industrial wastes containing heavy metals for removing inorganic pollution is achieved by using various types of microorganisms such as algae, bacteria, fibrous fungi and yeast cells that have the ability to separate, concentrate and block various metals.

The use of enzymes to improve the biological degradation of unwanted organic matter (hydrocarbons) in industrial wastes and sewage networks is well known. For instance, U.S. Pat. No. 3,635,797A1 recommends the use of naturally fermented organic material mixtures from certain yeast cultures, combined with surfactants and other biologically stimulating and/or preserving ingredients, to stimulate natural bacterial activities, catalyze the digestion of organic impurities, and eliminate offensive odors. It is also known that such a compound of fermentation reaction product improves the aeration and bacterial activities of the soil to which it is added and can be advantageously used against pests, as well.

The aim of an susceptible ore beneficiation process should be to achieve an efficient and sustainable operation of mineral deposits therethrough by utilizing the most suitable and effective technologies and converting them into valuable minerals by processing with the least possible loss, without having a negative impact on the environment and water resources. It is essential that mineral concentration processes and all natural fluids and resources, especially the water used herein, be used in the most effective and efficient way. Consequently, there always exists a need for value-added innovative studies with practical and environmentally-friendly approaches for ensuring the least waste of resources and the highest possible yield in the ore beneficiation process.

### OBJECTS AND BRIEF DESCRIPTION OF THE INVENTION

The main object of the present invention is to provide an environmentally-friendly biochemical composition for use as an additive into an aqueous medium in order to increase the process efficiency in all gravimetric and magnetic wet ore concentration methods that essentially require the use of water, in the mining industry.

It is another important object to provide a biochemical composition for increasing grade and yield in terms of target valuable mineral concentrate and ensuring a financially viable product, without requiring extra water usage, in specific gravity/gravity separation processes such as inclined flowing medium (shaking table, spiral, cyclone) and vertical moving medium (jig), and magnetic (low and high intensity) separation processes, that essentially require the use of water.

It is a further object to provide a biochemical composition for achieving more efficient operational conditions for the medium, equipment and machinery where water is employed and for minimizing the undesired contents, such as lime and silica, that complicate the operating and maintenance conditions.

In order to achieve the aforesaid objects, there is provided a cost-effective and environmentally-friendly biochemical liquid concentrate composition for use as an additive into the process/feed water or aqueous medium (in little amounts) in gravity and magnetic separation methods of ore beneficiation and classification processes requiring essentially the use of water, which provides an increase in grade and efficiency while reducing the environmental pollution. According to the present invention, the biochemical composition comprises a Saccharomyces cerevisiae yeast fermentation supernatant, one or more surfactants selected from the group consisting of non-ionic surfactants and anionic surfactants, at least one preservative agent, and preferably at least one oxidizing agent selected from the group consisting of hydrogen peroxide and chlorine dioxide, added in small amounts into the process/feed water or into the aqueous medium.

Said yeast supernatant can be produced using conventional fermentation methods known from the prior art, preferably with substantially-reduced or eliminated enzymatic activity and bacterial presence, or can be obtained in ready-made form. Said reduction or elimination processes can be carried out via autolyzing, heat treatment, pasteurization, denaturation of cells, addition of EDTA, and subjecting them to one or more of the appropriate chromatographic conventional applications, during the production stage of the supernatant.

The Saccharomyces cerevisiae yeast supernatant is present in the invention composition preferably at a concentration of 5% to 35%, by total weight.

Said surfactants can be selected from nonionic surfactants and anionic surfactants, or preferably from one or more nonionic surfactants, at a concentration of 5 to 35%, by weight. No cationic surfactant is available. The weight ratio of anionic surfactants, if any, to the total surfactant content is at most 20%, preferably ranging from 0.1% to 10%.

The nonionic surfactants can be selected from the group consisting of alkyl, aryl, polyethylene glycol ether, ethylene glycol, or any type of an ethylene oxide chain containing a water-soluble moiety, in particular nonylphenol ethoxylates, octylphenol ethoxylates, alkyl ethoxylates, ethoxylated amine salts, alkylphenol ethoxylates and other derivatives and mixtures thereof, having an ethoxylate moiety. Preferably, the nonionic surfactants comprise at least one to be selected from the group consisting of ethoxylated alcohol, ethoxylated aliphatic alcohol, amine oxide, alkylamine, ethoxylated alkylamine, ethoxylated alkyl phenol, alkyl polysaccharide, ethoxylated alkyl polysaccharide, and ethoxylated fatty acid. More preferably, it is selected from the group individually or in combination consisting of ethoxylated dodecyl alcohol, ethoxylated octyl phenol and tridecyl alcohol ethoxylate.

The anionic surfactants comprise at least one to be selected from the group consisting of alkylsulfonate, alkyldiphenyloxide disulfonate, alkylphenol polyoxyethylene ether phosphate ester and fatty alcohol polyoxyethylene ether sulfate, sodium lauryl sulfate, sodium dodecyl benzene sulfonate, sodium disulfonate, sodium dodecyl phosphate and sodium dodecylate.

The preservative agents are preferably selected from the group consisting of sodium benzoate, imidazolidinyl urea, diazolidinyl urea, polyoxymethylene urea, quaternium-15, DMDM hydantoin, bromopol, glyoxal, sodium hydroxymethylglycinate, alkyl paraben and glycerin, and are present in the composition at a concentration of 0.1% to 4.5%, by weight.

Said hydrogen peroxide and/or chlorine dioxide are/is present at a concentration of 0.1% to 1.5%, by weight.

The pH of the biochemical composition, which is completed to 100% with water, is at most 7.0, preferably 2.5 to 6.5, more preferably 3.0 to 5.5. The water may herein refer to mains water, tap water, softened water, filtered water, purified water, pure water, or a combination thereof.

In an alternative embodiment, a weak organic acid, preferably acetic acid, may be added to the composition at a concentration of 0.05 to 0.5%, by weight.

The composition may also comprise a sequestrant and/or stabilizer, i.e. EDTA, phosphonic acid, or a combination thereof.

The biochemical composition of the invention can be provided, preferably in the form of liquid concentrate of predetermined appropriate proportions, into the still or flowing aqueous medium by means of a suitable watering, dripping or spraying embodiment before or during the application of any wet process in ore beneficiation and classification processes. For example, feeding the invention composition preferably less than 1000 cc, more preferably less than 100 cc per tonne of water used, in other words, at a dilution ratio of 1:10.000 to 1:100.000 can be sufficient to achieve the aforesaid objects.

The biochemical composition of the invention can be successfully applied in inclined flowing medium (shaking table, spiral, cyclone), vertical moving medium (jig), and magnetic (low and high intensity) separation processes, that essentially require the use of water. According to the invention, a multi-purpose and versatile formulation of cost-effective and environmentally-friendly biochemical liquid concentrate composition is provided for use simply as an additive into the process feed water, due to the fact that it reduces the viscosity and surface tension, and increases the formation of microbubbles in the ore mixture, thus facilitating the separation of minerals on the one hand, and reducing the need for maintenance and downtime of the medium, equipment, and machinery where water is employed, by preventing lime formation, clogging and clumps, on the other hand.

The composition of the invention has no corrosive effect, is non-toxic and is 100% naturally soluble. Owing to the physical and physicochemical advantages thereof, higher operational efficiency and higher gains with appropriate operating conditions can be obtained. The supernatant obtained from S. cerevisiae yeast cells together with the composition content provides an unexpected technological added value since a very low amount of the same is added into water and such use is completely distinguished from the primary purposes and applications of the prior art, such as bioremediation of toxic metals from wastewater or treatment of hydrocarbon-containing wastes.

The supernatant, purified from plant origin and preferably with reduced or eliminated enzymatic activity and bacterial presence, has versatile and advantageous effects on lowering the surface tension due to its broad protein spectrum content. Together with non-ionic-anionic or preferably non-ionic biological surfactants and other ingredients of the invention, the said advantages are enhanced synergistically. Thus, the composition increases the dissolved oxygen in the medium, accelerates the separation process, and reduces the need for chemical use. Besides, it minimizes clogging and slime formation and reduces metal corrosion, as well.

These and other features, advantages, and embodiments of the present invention will become more apparent from, and will be understood more clearly by reference to, the following detailed description and the associated examples.

### DETAILED DESCRIPTION

Owing to the particular formulation and content ratios of the invention, there is provided a biochemical composition comprising a fermentation supernatant obtained from the culture of Saccharomyces cerevisiae, one or more surfactants selected from the group consisting of non-ionic surfactants and anionic surfactants, preferably hydrogen peroxide or chlorine dioxide, and urea-based or other suitable preservatives, which increases the efficiency synergistically and unexpectedly in gravity and magnetic wet separation methods using essentially water, without requiring an additional facility investment, in ore beneficiation and classification processes.

The use of supernatants obtained by fermentation from yeast cultures such as Saccharomyces cerevisiae, Kluyveromyces marxianus, Kluyveromyces lactis, Candida utilis, ZygoSaccharomyces, Pichia and Hansanula together with surfactants and preservatives is known from the prior art teachings for the removal of heavy minerals and hydrocarbons in industrial wastes, organic and odorous micro-organisms and heavy metals in sewage and wastewater, biofilm formation on surfaces and pests in agriculture.

Saccharomyces cerevisiae is also known as baker's yeast. Named among the most well-known and best-studied important yeast strains for use in beer, wine, and bread baking since ancient times, S. cerevisiae strains are described as “generally recognized as safe” organisms by the US Food and Drug Administration (FDA), which means that these cells can be freely manipulated without public concern. The culture of S. cerevisiae included in the composition of the invention and the preparation of supernatant thereof are also well known from the prior art.

For instance, U.S. Pat. No. 3,635,797A discloses that the yeast S. cerevisiae can be initially cultured in a medium containing a sugar source such as molasses, raw sugar, soybean, or sucrose consisting of mixtures thereof. The mixture of sugar, diastatic malt, S. cerevisiae yeast, and magnesium salt is incubated for two to five days at suitable temperatures (25 to 45° C.) until the fermentation is completed, and then the unwanted residues are separated, preferably by centrifugation, to obtain the supernatant. The patent document mentions the preparation of an aqueous enzymatic composition comprising an enzymatic fermentation reaction product, surfactants, citric and lactic acids, urea, and pine oil, for use in protein-surfactant systems directed to the applications of, for instance, mainly the removal of hydrocarbon-based wastes from petroleum products and industrial wastes as well as water and sewage treatment. Surfactants are selected from organic, anionic, and nonionic surfactants and inorganic alkali metal phosphates, borates, carbonates, silicates, or mixtures thereof.

The method for preparing the supernatant culture according to the invention may comprise the following basic steps: Fermenting and growing the yeast in a rich nutrient medium, then removing cells and residues by centrifugation, mixing the obtained fermentation supernatant with sodium benzoate, imidazolidinyl urea, diazolidinyl urea and/or other mentioned suitable agents, for instance, heating to 40-45° ° C., stirring for 1-2 hours to dissolve the components, then mixing the resulting intermediate with other ingredients such as surfactants and preservatives to obtain the final composition, and if necessary, adjusting the PH to e.g. 2.5. to 6.5, preferably 3.0 to 4.5.

For the inactivation and treatment of S. cerevisiae cells, various conventional methods known from the prior art can be applied, such as lyophilization, denaturation, pasteurization, autoclaving, irradiation, heat treatment, and chemical treatment with alkaline solutions, ethanol, formaldehyde, and acetone in order to reduce or eliminate enzymatic activity and bacterial presence therein. Likewise, the appropriate method may also be selected among the applications such as (heat) shocking via heating up to 50-70° C. for 2 to 24 hours—before or after centrifugation—and/or mechanical physical stressing (pressing, rolling, high-pressure homogenization), chemical degradation (alcohol extraction, EDTA addition, cell lysis) and/or anion exchange chromatography.

Since non-ionic surfactants are not affected by acidic or basic environments, they are the most widely used surfactants in detergent, cosmetics, and other similar industries. They are used widely and effectively in wetting, dispersing, and spreading and as emulsifiers, foaming (control) agents, cleaning agents for detergents, general cleaning agents, wetting agents in textile and pesticide formulations, and have compatibility with other surfactants.

In a preferred embodiment of the present invention, the final composition in concentrated liquid form comprise about 5% to 35% Saccharomyces Cerevisiae fermentation supernatant, about 5% to 35% nonionic surfactant, 1% to 7.5% anionic surfactant, about 0.1% to 1.5% hydrogen peroxide and/or chlorine dioxide, about 0.1% to 3.5% sodium benzoate, and/or about 0.001% to 0.04% imidazolidinyl urea and/or about 0.01% to 0.4% diazolidinyl urea, by weight. Also, a nitrogen source such as urea or ammonium nitrate can be added at a concentration of 3% to 30% by weight in the final composition.

If necessary, the pH of said composition can be adjusted with an acid such as citric acid or phosphoric acid to a maximum of 7.0, preferably 3.0 to 4.5.

As an example of another preferred embodiment, the biochemical liquid concentrate composition of the invention comprises, by weight, 25.0% S. cerevisiae supernatant, 11.25% ethoxylated octyl phenol, 1.25% alkyldiphenyloxide disulfonate, 0.20% diazolidinyl urea, 0.35% sodium benzoate, 0.15% hydrogen peroxide and water to add up to 100%. Depending on the wet process requirements for the ore beneficiation, said liquid concentrate composition may be provided with a dilution ratio of one thousandth to one millionth, preferably one ten-thousandth to one hundred-thousandth, per tonne of water, by volume, into the still or flowing aqueous medium.

In order to measure the efficiency of the composition in the wet beneficiation and classification processes, the first tests were carried out on the widely-used shaking tables. Beforehand, valuable minerals in the ore are liberated by size reduction by using conventional crushing and grinding methods and then classified in hydraulic classifiers (hydrosizers), when necessary, to optimize the grain sizes.

The shaking table is a slightly inclined, tilt-adjustable, parallelogram, rectangular-like trapezoidal table, having a sloping deck with a riffled/ribbed surface across which a film of water flows. There is a feeding box on the upper part and a wash water dispenser, which consists of a perforated chute.

The amount of water flowing through each hole is adjusted with latches, allowing the water on the table surface to flow in a single layer (film). The mineral grains of feed material on the deck surface move diagonally in the resultant direction of the movement of the water flow in a single layer (film) across the deck, and back and forth movement perpendicular thereto, as the heavy and lighter minerals simultaneously spread out and can be variously collected.

Physical conditions such as free flow, hindered settling, and asymmetrical movement are effective in the operation of the table. The heavy material is less subject to cross-flow forces than the lighter material so that the feed is differentiated into strips according to density with the heavy minerals discharging as concentrate over the end, middlings near the lower corner, and tailings over the long side.

### Efficiency Tests

In order to determine the effectiveness and efficiency of the invention composition, the gravity concentration of chromite ore was carried out using a wet shaking table at the Mineral Processing Pilot Facility of Istanbul Technical University (ITU) Mining Faculty, and all samples taken were analyzed in an accredited laboratory. The ore contains 10.28% Cr₂O₃ and 19.2% Al, by weight, with a particle size of about 2 mm.

In the said shaking table test, the invention composition was added into the feed water and the results were compared to the control group, which was carried out under exactly the same conditions. The viscosities of the running water were measured before and after the invention composition was added therein. Two different pulp water were prepared for use on the shaking table, the viscosity values of which are given in Table 1. The ore material was first concentrated with water. The second measurement was performed by adding the 0.13% solution of the aforesaid invention composition in to the water, by volume.

Due to the fact that these tests were carried out in experimental conditions using a laboratory shaking table, i.e. with 640×1280 mm of very small deck dimensions and running at a low capacity of 80 kg/hour, the invention composition was added into the feed water at a rate of 7 lt/min. The amplitude of the table was set to 10 mm and the deck tilt angle to 7º. The surface tension values obtained from the measurements with the Brookfield Viscometer are given in Table 1 below:

TABLE 1
Viscosity Comparison Values

Viscosity Value (N · sn/m²)

| RPM | Water | Water + Invention Composition Solution |
|---|---|---|
| 5 | 300 | 200 |
| 20 | 125 | 50 |
| 50 | 45 | 30 |

In the measurements obtained by the Du Nouy ring method, it was observed that the surface tension decreased by more than half (from 72.8 to 34.5 dyne/cm) with the addition of the invention composition solution into the feed water.

Since the parameters such as the ore dimension (grain size), velocity (stroke frequency) and amplitude (stroke length), solid ratio of the feed material and slope (tilt angle) were effective for the capacity and performance of the operations, they were all kept constant in all tests in order to reach an accurate and reliable comparison. In these shaking table tests performed under exactly the same conditions, gain differences as a result of said two applications, i.e. use of the tap water as the control group and use of the same together with the invention composition solution as an additive, are reported in Table 2.

TABLE 2
Efficiency Comparison Values

| Application | | Cr₂O₃ (wt %) | Efficiency (%) |
|---|---|---|---|
| Test 1a | Concentrate | 33.61 | 58.5 |
| (water) | Intermediate | 21.31 | |
| | Gangue/Waste | 5.39 | |
| | Feed | 10.33 | |
| Test 1b | Concentrate | 33.40 | 60.9 |
| (Water + Invention | Intermediate | 23.75 | |
| Composition Solution) | Gangue/Waste | 5.47 | |
| | Feed | 10.33 | |

In these tests, the position of the wash water dispenser was kept constant. Therefore, the increased grade was not collected in the concentrate but spread to the intermediate product and gangue. As a result, there was an increase in the grade and accordingly a more efficient concentration, i.e. +2.4%, was observed.

Yet, a similar test was repeated during actual operations of a beneficiation plant by using the ore material from the same mineral deposit. 0.01% of the invention composition solution, by volume, was added into the feed water of the Wilfley-type shaking table by means of a dripper, from the hydrosizer outlet. The basic parameters and the results are reported in Tables 3 and 4 below.

TABLE 3
Table operation parameters

| Table Performance | Values | |
|---|---|---|
| Grain Size | −0.7 + 0.4 | mm |
| Capacity | 0.65 | ton/hr. |
| Amplitude | 15 | mm |
| Frequency (Speed) | 300 | rpm |
| Table Size | 1.95 × 4.8 | m |

| | |
|---|---|
| Table slope (tilt) | %0.90 |

TABLE 4
Ore measurement results

| Pulp-Solid | Water | Water + Invention Solution |
|---|---|---|
| Ratios | (% Cr₂O₃ wt.) | (% Cr₂O₃ wt.) |
| Feed | 10.35 | 10.35 |
| Concentrate | 34.92 | 36.87 |
| Intermediate | 30.22 | 34.36 |

| | |
|---|---|
| Gangue/Waste | Not measured. |

| | | |
|---|---|---|
| Water Usage | 2.3 m³/hr | 2.3 m³/hr |

As being apparent from the results, the yield obtained with the addition of the invention solution shows an increase of 5.6% in the concentrate and 13.7% in the intermediate product, compared to the use of water alone.

With the use of the invention composition in these wet processes, it was also observed that the slime decomposed more easily from the ore material and the ratio of silica content decreased. It prevented lime formation, clogging, and clumps, and reduced problems such as excessive slime formation and metal corrosion. For instance, such formations reduce the process efficiency as they reduce the efficiency of the deck surface and result in the need to slow down the shaking table operation. Owing to the purified broad protein spectrum of the composition, water is diluted more effectively, and high synergistic supernatant activation is provided with the surfactant, thus eliminating the said inconvenience and further reducing the requirements for maintenance and downtime.

On the other hand, the beneficiation of some ores is carried out by taking advantage of the difference in magnetic and electrostatic physical characteristics of the minerals they specifically have. Magnetic separation utilizes the force of a magnetic field to produce differential movement of mineral particles through a magnetic field; gravitational and frictional forces also help for the separation thereof. The principle used in the concentration of para- and especially ferro-magnetic minerals is that an inhomogeneous magnetic field is provided and magnetic minerals move towards a point of the strongest magnetic field, while non-magnetic minerals remain inactive.

Depending on the magnetic properties of the minerals, various machines can be used, i.e. low-intensity separators for concentrating ferro-magnetic minerals and high-intensity separators for para-magnetic minerals. Some iron minerals of low magnetic properties such as hematite and limonite are first roasted to increase their magnetization, and then concentrated by means of low-intensity separators. Para-magnetic minerals such as ilmenite, rutile, wolframite, monazite, siderite, chromite, hematite and manganese are separated by high-intensity magnetic separators.

The product obtained as a result of the shaking table application given in Table 4 above, was subjected to the magnetic separation test performed under exactly the same conditions compared to the use of water alone. With the application of a 5 cc solution of the invention composition per tonne of the feed water, the weight ratio of chromium to iron in the separator outlet reached from 1.89% to 2.22%. Therefore, the yield in the concentrate obtained by adding the invention solution into the process water increases by 17.5% compared to the application of water alone.

In another study aiming to reduce iron oxide content of quartzites, given in Table 6 below, down to the target limit of 0.05% for use in glass production, the invention composition was tested by adding the same into the feed water of high-intensity wet magnetic separator under laboratory conditions and compared to the application of water alone under the same conditions.

TABLE 5
Run-Of-The Mine Ore Chemical Content Analysis

| Content | Weight % |
|---|---|
| SiO₂ | %97.35 |
| Al₂O₃ | %1.66 |
| Fe₂O₃ | %0.16 |
| TiO₂ | %0.13 |
| MgO | %0.07 |

TABLE 6
Test Parameters

| Parameter | Value | |
|---|---|---|
| Pulp-Solid Ratio | %10 | |
| Feed Rate | 3 | kg/sn |
| Magnetic Field | 19.000 | Gauss |
| Gangue/Waste | 23.79 | |
| Grain Size | −0.21 + 0.11 | mm |

After being classified by crushing and grinding with conventional methods, the sample in pulp form was processed in a laboratory type, wire matrix, high-intensity wet magnetic separator. By keeping the parameters given in Table 6 constant, a Fe₂O₃ concentrate of 0.049%, by weight, was obtained with a residue removal efficiency of 76.23%, by using the water alone, whereas a Fe₂O₃ concentrate of 0.041% in grade, by weight, was obtained with a residue removal efficiency of 77.03%, by using the water and the invention composition added therein in the range of %0.05. This test reveals the fact that invention composition contributes 16.3% as an increase in grade.

Likewise, owing to the invention composition, the viscosity of the aqueous medium is effectively reduced, the amount of dissolved oxygen is increased, and there is provided the formation of microbubbles in the ore mixture. Therefore, the separation of minerals are facilitated in a more efficient way compared to the use of water alone. A synergistic effect is also achieved in terms of increased efficiency, grade and value, together with the reasonable and accurate use of surfactants, which are generally not preferred in terms of quantity, cost, and environmental issues in such processes under ordinary conditions. It is observed that it accelerates the separation/decomposition activities in the medium where it is added, and reduces the need for chemical use, if any.

While certain examples and embodiments of the present invention have been described so far, it is apparent to those skilled in the art that various other changes and modifications can be made without departing from the spirit and scope of the invention. Therefore, with the attached claims, it is ensured that such changes and modifications are included in the scope of protection without departing from the scope and integrity of the invention.

1. A biochemical liquid concentrate composition for use as an additive into the process water or feed water in gravity and magnetic separation methods of ore wet beneficiation and classification processes that essentially require the use of water, comprising
Saccharomyces cerevisiae yeast fermentation supernatant at a concentration of 5 to 35%, by weight;
one or more surfactants selected from the group consisting of nonionic surfactants and anionic surfactants at a concentration of 5 to 35%, by weight; and
at least one preservative agent selected from the group consisting of sodium benzoate, imidazolidinyl urea, diazolidinyl urea, polyoxymethylene urea, quaternium-15, DMDM hydantoin, bromopol, glyoxal, sodium hydroxymethylglycinate, alkyl paraben, and glycerin at a concentration 0.1% to 4.5%, by weight.

2. The composition according to claim 1, further comprising at least one oxidizing agent selected from the group consisting of hydrogen peroxide and chlorine dioxide at a concentration of 0.1 to 1.5%, by weight.

3. The composition according to claim 1, wherein the weight ratio of said anionic surfactants to the total surfactant content is at most 20%, preferably ranges from 0.1% to 10%.

4. The composition according to claim 1, wherein the yeast supernatant produced by fermentation from the Saccharomyces cerevisiae culture has reduced or eliminated enzymatic activity and bacterial presence.

5. The composition according to claim 1, wherein said nonionic surfactant content comprises at least one surfactant selected from the group consisting of amine oxide, ethoxylated alcohol, ethoxylated aliphatic alcohol, alkylamine, ethoxylated alkylamine, ethoxylated alkyl phenol, alkyl polysaccharide, ethoxylated alkyl polysaccharide, and ethoxylated fatty acid.

6. The composition according to claim 5, wherein said nonionic surfactant content comprises at least one surfactant selected from the group consisting of ethoxylated dodecyl alcohol, ethoxylated octyl phenol, and tridecyl alcohol ethoxylate.

7. The composition according to claim 1, wherein said anionic surfactant content comprises at least one surfactant selected from the group consisting of alkylsulfonate, alkyldiphenyloxide disulfonate, alkylphenol polyoxyethylene ether phosphate ester, and fatty alcohol polyoxyethylene ether sulfate, sodium lauryl sulfate, sodium dodecyl benzene sulfonate, sodium disulfonate, sodium dodecyl phosphate, and sodium dodecylate.

8. The composition according to claim 1, further comprising a weak organic acid, preferably acetic acid, at a concentration of 0.05 to 0.5%, by weight.

9. The composition according to claim 1, further comprising a nitrogen source having urea or ammonium nitrate at a concentration of 3 to 30%, by weight.

10. The composition according to claim 1, further comprising EDTA, phosphonic acid, or a combination thereof as a sequestrant and/or stabilizer.

11. The composition according to any one of the preceding claims, claim 1, characterized by having a pH below 7.0, preferably between 2.5 and 6.5.

12. The composition according to claim 1, characterized by providing the same with a dilution ratio of one thousandth to one millionth, preferably one ten-thousandth to one hundred-thousandth, per tonne of water, by volume, into a still or flowing aqueous medium used before or during said wet process.

13. A method for increasing grade and yield in any gravity and magnetic separation methods of ore wet beneficiation and classification processes that essentially require the use of water, comprising adding the composition according to claim 12 into the process water or feed water, thereby effectively reducing the viscosity of the aqueous medium, providing microbubble formation in the ore mixture, increasing the amount of dissolved oxygen, accelerating the decomposition process and facilitating the separation of minerals in a more efficient way compared to the use of water alone.

14. The method according to claim 13 wherein said wet gravity separation process is carried out by means of a vertically moving medium such as a jig that essentially uses water.

15. The method according to claim 13, wherein said wet gravity separation process is carried out by means of a layered, inclined flowing medium, such as a shaking table, spiral, or pinched sluices, that essentially uses water.

16. The method according to claim 13, wherein said magnetic separation process is carried out by means of low- or high-intensity wet magnetic separators that essentially use water.

17. The composition according to claim 12, wherein the composition is fed into the still or flowing aqueous medium by means of a suitable watering, dripping or spraying embodiment before or during the application of any wet process in ore beneficiation and classification processes at preferably less than 1000 cc and more preferably less than 100 cc per tonne of water used.
